(19) United States
(12) Patent Application Publication
(10) Pub. No.: US 2026/0325833 A1
(43) Pub. Date: Oct. 22, 2026

(54) TEMPORARY PACING LEAD MANAGEMENT SYSTEM

(71) Applicant: Medtronic, Inc., Minneapolis, MN (US)

(72) Inventors: Mikayle A. Holm, Saint Anthony, MN (US); Kevin R. Seifert, Forest Lake, MN (US); Jonathan A. Hughes, Blaine, MN (US); Thomas A. Wong, Little Canada, MN (US)

(21) Appl. No.: 19/114,731

(22) PCT Filed: Sep. 18, 2023
(86) PCT No.: PCT/US2023/074473

§ 371 Date: Mar. 24, 2025

Related U.S. Application Data
(60) Provisional application No. 63/377,143, filed on Sep. 26, 2022.

(51) Int. Cl. A61N 1/05; A61N 1/362

(52) U.S. Cl. CPC A61N 1/057; A61N 1/362

(57) ABSTRACT

This disclosure describes a system comprising an electrical lead, an implantable medical device, wherein the electrical lead is configured to be electrically connected to the implantable medical device, and the implantable medical device is configured to deliver an electrical therapy to tissue of a patient via the electrical lead, and an expandable member configured to be disposed over the implantable medical device and an excess portion of the electrical lead, wherein the expandable member is configured to control movement of the electrical lead within vasculature of the patient.

This application claims the benefit of U.S. Provisional Patent Application No. 63/377,143, filed Sep. 26, 2022 and entitled “TEMPORARY PACING LEAD MANAGEMENT SYSTEM,” the entire contents of which is incorporated herein by reference.

### TECHNICAL FIELD

This disclosure relates to medical device systems and, more particularly, to medical device systems for delivery of electrical stimulation therapy.

### BACKGROUND

In some situations, medical professionals may insert an implantable medical lead into the body of the patient to deliver various therapies. In some examples, the implantable medical leads may be temporary and may be removed from the patient after a period of time. In some examples, with respect to cardiac-related issues, a medical professional may insert a type of implantable medical lead also referred to as a temporary pacing lead into a heart of the patient and deliver temporary cardiac pacing to the heart.

### SUMMARY

The devices, systems, and techniques of this disclosure generally relate to managing movement of an implantable medical lead within a patient. In some examples, the implantable medical lead may have an excess portion that may be placed near an implantable medical device connected to the implantable medical lead. A clinician may need to control movement of the implantable medical lead within the patient. In some examples, the clinician may need to remove the implantable medical device and the implantable medical lead from the patient. This disclosure describes devices, systems, and techniques for managing movement of the implantable medical lead within the patient and devices, systems, and techniques for facilitating removal of the implantable medical lead from the patient.

In an example, the disclosure describes a system comprising: an electrical lead; an implantable medical device, wherein the electrical lead is configured to be electrically connected to the implantable medical device, and the implantable medical device is configured to deliver an electrical therapy to tissue of a patient via the electrical lead; and an expandable member configured to be disposed over the implantable medical device and an excess portion of the electrical lead, wherein the expandable is configured to control movement of the electrical lead within vasculature of the patient.

In another example, the disclosure describes a lead management device comprising: an expandable member; and an inner volume defined by the expandable member, the inner volume configured to retain an implantable medical device configured to deliver an electrical therapy to tissue of a patient and an excess portion of an electrical lead electrically connected to the implantable medical device, wherein the expandable member is configured to control movement of the electrical lead within vasculature of the patient.

In another example, the disclosure describes a method comprising: inserting a distal portion of an electrical lead into vasculature of a patient; navigating the distal portion of the electrical lead to a target location within the vasculature of the patient; implanting the distal portion of the electrical lead in tissue of the patient at the target location; disposing an implantable medical device and an excess portion of the electrical lead within an expandable member, wherein the expandable member is configured to control movement of the electrical lead; and delivering electrical therapy to the tissue at the target location through the electrical lead.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a conceptual diagram illustrating an implantable medical device system including a lead management system.

FIG. 2 is a block diagram illustrating an example configuration of an implantable medical device of the system of FIG. 1.

FIG. 3A is a conceptual diagram illustrating an example lead management system in a pre-deployed configuration.

FIG. 3B is a conceptual diagram illustrating the example lead management system of FIG. 3A in a deployed configuration.

FIG. 4A is a conceptual diagram illustrating a side view of an example retainer of an example lead management system.

FIG. 4B is a conceptual diagram illustrating a top-down view of the retainer of FIG. 4A.

FIG. 4C is a conceptual diagram illustrating the retainer of FIG. 4A with an implantable medical device and an expandable member in a pre-deployed configuration.

FIG. 5 is a conceptual diagram illustrating another example retainer of an example lead management system.

FIG. 6A-D are conceptual diagrams illustrating an example process of implanting an expandable member and an implantable medical device into tissue of a patient via the retainer of FIG. 5.

FIG. 7 is a flowchart illustrating an example process of delivering electrical signals to a patient.

FIG. 8 is a flowchart illustrating an example process of implanting an expandable member and an implantable medical device in a patient.

### DETAILED DESCRIPTION

Medical devices, systems, and techniques of this disclosure relate to managing movement of an implantable medical lead within a patient and facilitating removal of the implantable medical lead from the patient. A medical professional may insert an implantable medical lead into the patient (e.g., through vasculature of the patient) to deliver electrical signals (e.g., for purposes of medical therapy, stimulation, or the like) to tissue of the patient. The implantable medical lead (also referred to herein as “electrical lead”) may be connected to an implantable medical device. The implantable medical device may generate the electrical signals and transmit the electrical signals to the tissue of the patient through one or more implantable medical leads.

In some examples, based on the anatomy of the patient and/or the target location, the length of the implantable medical lead may be longer than necessary to reach the target site from the implantable medical device, and the clinician may store the excess lead length in subcutaneous tissue of the patient (e.g., adjacent to the implantable medical device within the subcutaneous tissue). Storage of the excess lead length in the tissue of the patient may lead to twisting, crimping, and/or pinching of the implantable medical lead which may negatively affect performance of the lead. In some examples, tissue may grow around the implantable medical device and/or the excess lead lengths within the subcutaneous tissue and renders removal of the implantable medical device and/or the implantable medical lead difficult and time consuming.

This disclosure provides improvements over other implantable medical lead systems and insertion processes by allowing a clinician to manage the length of an implantable medical lead within the vasculature of the patient and/or to manage movement of the implantable medical lead within a subcutaneous pocket in the tissue of the patient and/or within the vasculature of the patient. The medical devices, systems, and techniques of this disclosure also isolate the excess portions of the implantable medical lead stored in the subcutaneous pocket from tissue of the patient and facilitate removal of the implantable medical device and the implantable medical lead during a later operation. The medical devices, systems, and techniques of this disclosure may also enable identification of an orientation of the distal end of the implantable medical lead in addition to its position.

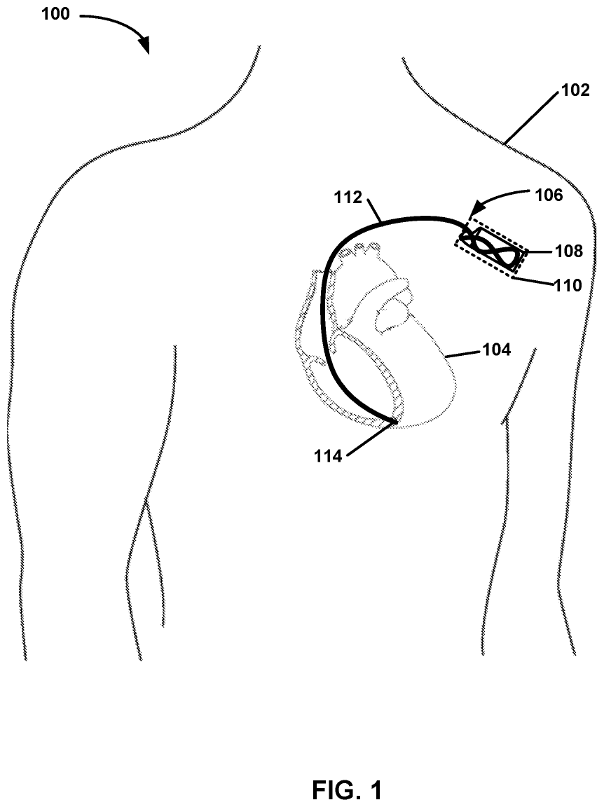

FIG. 1 is a conceptual diagram illustrating an implantable medical device system 100 including a lead management system 106. Medical device system 100 may include implantable medical device (IMD) 108, lead management system 106 including lead management device 110, and implantable medical lead 112 (also referred to herein as “electrical lead 112”). In some examples, as illustrated in FIG. 1, at least parts of medical device system 100 (e.g., IMD 108, lead management system 106, and electrical lead 112.) may be implanted within patient 102 (e.g., within a subcutaneous pocket in patient 102). A distal end 114 of lead 112 may be implanted at a target location (e.g., within heart 104 as illustrated in FIG. 1) within patient 102. In some examples, medical device system 100 may include a medical device disposed outside of the body of patient 102 and connected to electrical lead 112 implanted within patient 102 (e.g., at the target region within heart 104).

IMD 108 may include one or more devices configured to generate and deliver electrical energy (e.g., electrical stimulation, electrical therapy, or the like) to tissue of patient 102. The IMD 108 may be implanted within a subcutaneous pocket in patient 102. The subcutaneous pocket may be outside vasculature of patient 102. IMD 108 may deliver electrical energy to one or more target locations within patient 102 via one or more electrical leads 112. IMD 108 may be an implantable pulse generator (IPG), an implantable cardioverter defibrillator (ICD), a cardiac resynchronization therapy (CRT) device, or the like.

Electrical lead 112 may be connected to IMD 108 (e.g., to a distal end of IMD 108). In some examples, electrical lead 112 is fixedly attached to IMD 108, e.g., with no removable connection. In some examples, electrical lead 112 is removably attached to IMD 108, e.g., via a removable connector. Electrical lead 112 may transmit electrical signals between tissue of patient 102 and IMD 108. In some examples, as described in greater detail below, medical device system 100 may include a single electrical lead 112. In other examples, medical device system 100 may have two or more electrical leads 112. A clinician may insert electrical lead 112 into patient 102 at an insertion site (not pictured). The insertion site may be in the upper thorax or clavicular area of patient 102. In other examples, the insertion site may be at other locations on patient 102 (e.g., the groin, the neck, the abdomen, the arm, and the like). The medical professional may insert distal portion 114 of electrical lead 112 into vasculature of patient 102 at the insertion site and navigate electrical lead 112 to a target location (also referred to herein as “target treatment site”) within patient 102 through vasculature of patient 102 (e.g., within heart 114).

The clinician may navigate distal portion 114 of electrical lead 112 through the vasculature of patient 102 using one or more imaging techniques (e.g., X-ray imaging, fluoroscopy, ultrasound or the like). Once the clinician determines that distal portion 114 of electrical lead 112 is at the target location, the clinician may implant the distal portion 114 at the target location via one or more fixation mechanisms attached to distal portion 114 of electrical lead 112. Depending on the anatomy of patient 102 and/or distance within the vasculature between the target location and the insertion site, a proximal portion of electrical lead 112 (also referred to as “excess portion”) may be outside of the vasculature of patient 102.

Lead management system 106 includes lead management device 110 which may be disposed over IMD 108 and electrical lead 112 (e.g., the excess portion of electrical lead 112). Lead management device 110 (also referred to as “expandable member 110”) may be an expandable material and may control (e.g., inhibit or prevent) movement of electrical lead 112 within the vasculature of patient 102 by retention of the excess portion of electrical lead 112. Lead management device 110 containing IMD 108 and electrical lead 112 may be implanted within a subcutaneous pocket at the insertion site. While implanted, lead management device 110 may isolate IMD 108 and/or the excess portions of electrical lead 112 from tissue of patient 102 around the subcutaneous pocket and prevent tissue ingrowth around IMD 108 and/or electrical lead 112.

When the clinician decides to remove IMD 108 and/or electrical lead 112 from patient 102, the clinician may re-open the subcutaneous pocket and remove lead management device 110 containing IMD 108 and electrical lead 112 without necessitating removal of ingrown tissue around IMD 108 and/or electrical lead 112. In some examples, electrical lead 112 may remain implanted within patient 102 and the clinician may replace IMD 108 of medical device system 100 with another implantable medical device.

Medical system 100 may also include one or more computing devices and/or systems (not pictured) in communications with IMD 108. The one or more computing devices and/or systems may be operated by a user (e.g., by patient 102 and/or the clinician) and may transmit instructions to IMD 108. In some examples, the one or more computing devices and/or systems may receive patient information detected by IMD 108 (e.g., via electrical lead 112).

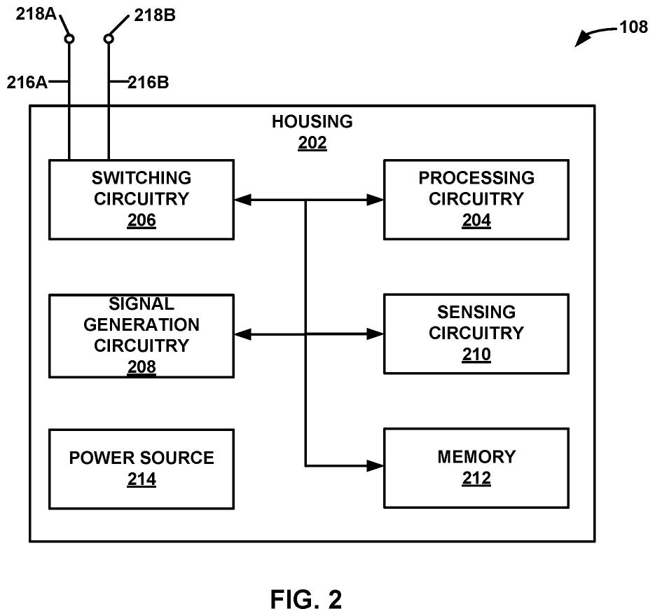

FIG. 2 is a block diagram illustrating an example configuration of an IMD 108 of the medical device system 100 of FIG. 1. IMD 108 may include processing circuitry 204, switching circuitry 206, signal generation circuitry 208, sensing circuitry 210, memory 212, power source 214, and electrodes 218A-B (also referred to as “electrodes 218”) connected to conductors 216 A-B (also referred to as “conductors 216”). Conductors 216 and electrodes 218 may be disposed within electrical lead 112 (not pictured). While FIG. 2 illustrates IMD 108 having two electrodes 218, other example IMDs may include three or more electrodes, each electrode connected to a corresponding conductor. In some examples, IMD 108 may have additional components (e.g., communications circuitry or the like). In some examples, as illustrated in FIG. 2, the components of IMD 108 may be contained within housing 202. In some examples electrodes 218 may be disposed in distal end 114 of electrical lead 112.

The various circuitry may be, or include, programmable or fixed function circuitry configured to perform the functions attributed to respective circuitry. Memory 212 may store computer-readable instructions that, when executed by processing circuitry 204, cause computing system 112 and/or implantable medical lead 104 to perform various functions. Memory 212 may be a storage device or other non-transitory medium.

Signal generation circuitry 208 is configured to generate electrical signals (e.g., stimulation signals such as cardiac pacing pulses). Signal generation circuitry 208 may include, as examples, current or voltage sources, capacitors, charge pumps, or other signal generation circuitry. Switching circuitry 206 is coupled to electrodes 218 and may include one or more switch arrays, one or more multiplexers, one or more switches (e.g., a switch matrix or other collection of switches), one or more transistors, or other electrical circuitry. Switching circuitry 206 is configured to direct electrical signals from signal generation circuitry 208 to a selected combination of electrodes 218, having selected polarities, e.g., to selectively deliver electrical signals to one or more target locations within patient 102. In some examples, signal generation circuitry 208 and switching circuitry 206 may be configured to selectively deliver electrical signals to one or more chambers of heart 104 of patient 102 to deliver cardiac pacing pulses to heart 104.

Switching circuitry 206 may also selectively couple sensing circuitry 210 to selected combinations of electrodes 218 to selectively sense the electrical activity in one or more target locations within patient 102. Sensing circuitry 210 may include filters, amplifiers, analog-to-digital converts, or other circuitry configured to sense electrical signals (e.g., cardiac electrical signals) via electrodes 218. For example, switching circuitry 206 may couple each of electrodes 218 to respective sensing channels provided by sensing circuitry 210 to sense the electrical signals in the corresponding target locations. Sensing circuitry 210 may sense electrical signals via electrodes 218 and store the sensed electrical signals within memory 212.

Processing circuitry 204 may include any one or more of a microprocessor, a controller, a digital signal processor (DSP), an application specific integrated circuitry (ASIC), a field-programmable gate array (FPGA), discrete logic circuitry, or any other processing circuitry configured to provide the functions attribute to processing circuitry 204 herein may be embodied as firmware, hardware, software, or any combination thereof.

Processing circuitry 204 may retrieve and execute instructions from memory 212 and transmit instructions to switching circuitry 206, signal generation circuitry, and sensing circuitry 210 to perform the functions described above.

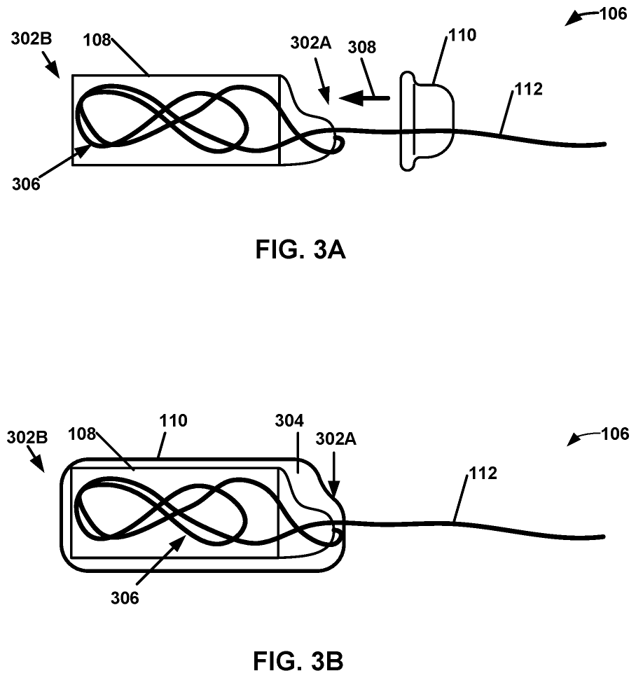

FIG. 3A is a conceptual diagram illustrating lead management system 106 in a pre-deployed configuration. As illustrated in FIG. 3A, lead management system 106 may include expandable member 110. expandable member 110 may be in a pre-deployed configuration before application over IMD 108. Expandable member 110 may be disposed over IMD 108 and excess portion 306 of electrical lead 112.

Expandable member 110 includes an expandable material configured to expand around and IMD 108 and excess portion 306. In some examples, the expandable material may include a biocompatible polymer including silicone, latex, polyurethane, or the like. Expandable member 110 may be transparent or translucent. The transparency of the expandable member 110 may allow the clinician to determine the positioning of IMD 108 and excess portion 306 within expandable member 110 and the presence of any twisting and/or crimping of any portion of electrical lead 112.

In some examples, expandable member 110 may include a relatively lubricious material disposed around an outer surface of expandable member 110. The lubricious material may increase the ease of insertion and/or extraction of expandable member 110 from the subcutaneous pocket by the clinician during medical device system 100 implantation and/or removal procedures. The lubricious material may include, but is not limited to, silicone oil, Polytetrafluoroethylene (PTFE), a calcium carbonate coating, or Ethylene tetrafluoroethylene (ETFE). Expandable member 110 may also include an anti-infection material disposed on an inner surface of expandable member 110. The anti-infection material may reduce adverse reactions by patient 102 to implantation of IMD 108, expandable member 110, and electrical lead 112 within tissue of patient 102. In some examples, the anti-infection material includes, but is not limited to, antimicrobial agents such as minocycline and/or rifampin. In some examples, expandable member 110 may include an anti-bleeding and anti-coagulation material. The anti-bleeding and anti-coagulation material may include, but are not limited to, Alginate, or Tranexamic Acid.

Expandable member 110 may transition between a pre-deployed configuration and a deployed configuration. In the pre-deployed configuration, expandable member 110 may be rolled over itself to include a folded section. Expandable member 110 may transition from the pre-deployed configuration to the deployed configuration by rolling the folded section towards the proximal end of expandable member 110. In some examples, expandable member 110 is configured to expand along a longitudinal axis of expandable member 110 and radially outwards of the longitudinal axis.

Expandable member 110 may define a distal opening and a proximal opening. The distal opening may allow for disposal of electrical lead 112 through expandable member 110 and into the vasculature of patient 102. The proximal opening may allow for disposal of IMD 108 and excess portion 306 of electrical lead 112 within an inner volume of expandable member 110.

The clinician may dispose expandable member 110 distal to distal end 302A of IMD 108. Electrical lead 112 may be disposed through expandable member 110 (e.g., through the distal opening of expandable member 110). In some examples, as illustrated in FIG. 3A, excess portion 306 may be arranged in a storage configuration and may be placed next to IMD 108. The storage configuration of excess portion 306 may be configured to organize excess portion within a defined space without crimping, twisting, pinching, or otherwise damaging electrical lead 112. In some examples, IMD 108 may include one or more fixation devices disposed on housing 202 that are configured to secure excess portion 306 (e.g., in the storage configuration) to housing 202 of IMD 108. In other examples, IMD 108 may be held together with excess portion 306 (e.g., via compressive force) until expandable member 110 is disposed over IMD 108 and excess portion 306.

The clinician may apply force 308 to roll expandable member 110 from the pre-deployed configuration to the deployed configuration. In some examples, the clinician may roll expandable member 110 from distal end 302A of IMD 108 to proximal end 302A of IMD 108.

Expandable member 110 may include one or more anti-rotation features configured to prevent rotation of IMD 108 within expandable member 110. By preventing rotation of IMD 108 within expandable member 110, the one or more anti-rotation feature may may prevent unintended dislodgement of electrical lead 112 within patient 102. The one or more anti-rotation features may include, but are not limited to, one or more protrusions extending from an inner surface of expandable member 110 and towards IMD 108, texturing on the inner surface of expandable member 110, or one or more other features configured to engage with a portion of housing 202 of IMD 108.

In some examples, the distal opening of expandable member 110 may be connected to a sheath (not pictured). The sheath may be configured to retain electrical lead 112. The sheath may be at least partially disposed within the vasculature and/or may be configured to anchor electrical lead 112 within the vasculature of patient 102. In some examples, the sheath may include one or more valves configured to prevent flow of fluids (e.g., blood of patient 102) between the vasculature of patient 102 and the subcutaneous pocket.

Examples of the sheath are described in the U.S. Provisional Patent Application entitled “Temporary Pacing Lead Management System”, having Attorney Docket Number A0007885US01/2222-205USP1 and filed on the same day as this disclosure, the entirety of which is incorporated by reference herein.

FIG. 3B is a conceptual diagram illustrating lead management system 106 of FIG. 3A in a deployed configuration. In some examples, as illustrated in FIG. 3B, expandable member 110 is deployed such that IMD 108 and excess portion 306 of electrical lead 112 are disposed within inner volume 110 of expandable member 110. In the deployed configuration, proximal end of expandable member 110 may constrict at a point proximal to proximal end 302B of IMD 108 and proximal opening of expandable member 110 may reduce in diameter and/or close.

When IMD 108 and excess portion 306 are disposed within inner volume 304, expandable member 110 may isolate IMD 108 and excess portion 306 from tissue of patient 102. Expandable member 110 may prevent tissue of patient 102 from growing into the recesses between IMD 108 and excess portion 306 and amongst the one or more coils of excess portion 306. The clinician may then implant lead management system 106 in the deployed configuration into the subcutaneous pocket in tissue of patient 102.

While IMD 108 excess portion 306 is disposed within inner volume 304, expandable member 110 may compress against IMD 108 and excess portion 306 (e.g., due to elastic properties of the expandable material of expandable member 110). Excess portion 306 may be compressed against IMD 108 and expandable member 110, which may prevent movement of electrical lead 112 into and/or out of the vasculature of patient 102 and/or expandable member 110.

In some examples, as illustrated in FIGS. 3A and 3B, expandable member 110 is rolled from distal end 302A of IMD 108 towards proximal end 302B of IMD 108. In some examples, expandable member 110 may be rolled from proximal end 302B towards distal end 302A.

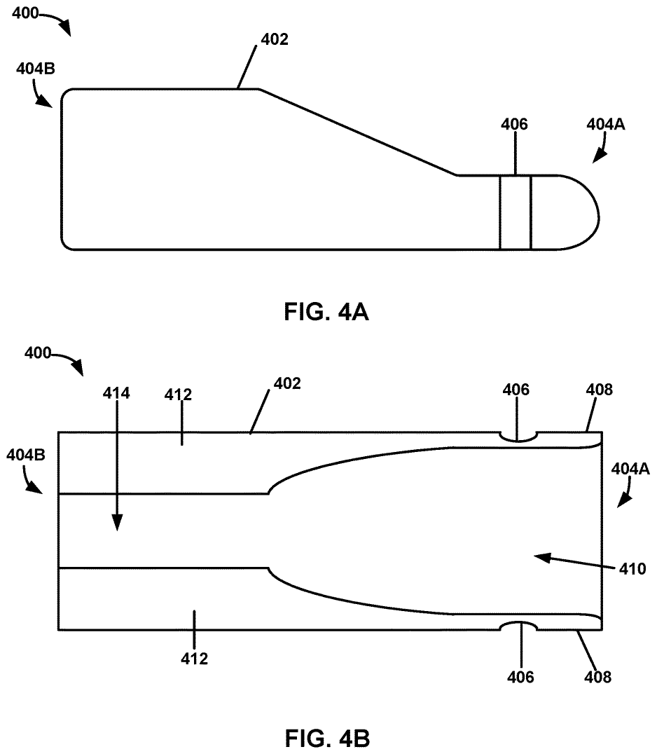

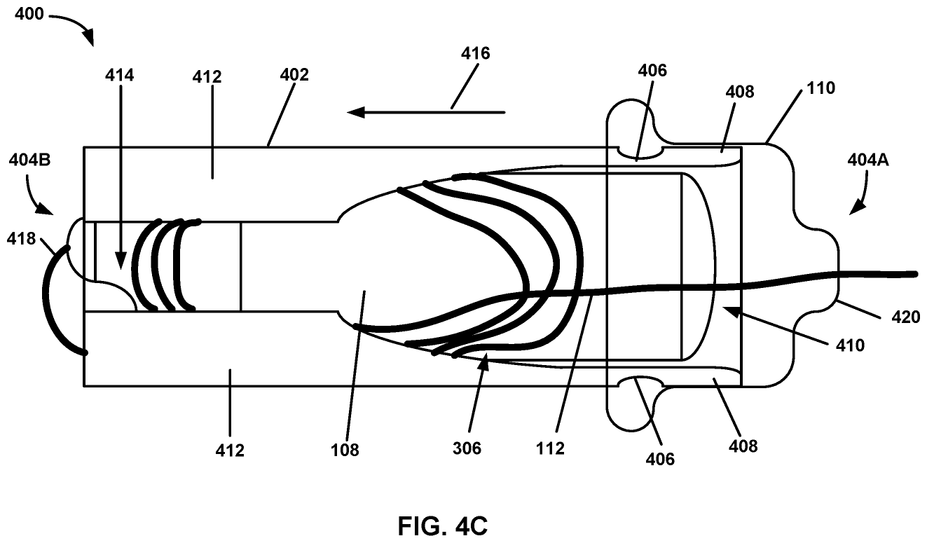

FIGS. 4A and 4B are conceptual diagrams illustrating a side view and a top-down view of a retainer 400 of lead management system 106, respectively. As illustrated in FIGS. 4A and 3B, retainer 400 may include a retainer body 402 defining a distal end 404A and a proximal end 404B. retainer body 402 further includes a plurality of flanges 408 extending towards distal end 404B, a plurality of recesses 406 defined by flanges 408, a plurality of flanges 412, a recess 410 (also referred to as “channel 410”) defined by flanges 408 and flanges 412 and extending from distal end 404A to proximal end 404B, and an opening 414 between flanges 412. Retainer 400 may facilitate deployment of expandable member 110 over IMD 108 and excess portion 306 of electrical lead 112.

Retainer 400 may include one or more biocompatible polymer including, but are not limited to, Acrylonitrile Butadiene Styrene (ABS), Polyether Ether Ketone (PEEK), Polycarbonates, Polyetherimide (PEI), Polysufone, or Polyurethane. Retainer 400 may be constructed using any of a plurality of known manufacturing methods including, but are not limited, to casting, molding, additive manufacturing, or 3D printing.

The clinician may insert IMD 108 and excess portion 306 into retainer 400, e.g., from distal end 404A. Retainer 400 may retain IMD 108 and excess portion 306 in recess 410. Flanges 408 and flanges 412 may prevent IMD 108 and/or excess portion 306 from moving within recess 410. Opening 414 may allow the clinician to determine a position and/or orientation of IMD 108 and/or excess portion 306 within retainer 400 and to reposition IMD 108 and/or excess portion 306 within retainer 400 if necessary.

FIG. 4C is a conceptual diagram illustrating the retainer of FIG. 4A with an IMD 108 and expandable member 110 in a pre-deployed configuration. As illustrated in FIG. 4C, expandable member 110 is retained on retainer 400 by recesses 406. IMD 108 and excess portion 306 is disposed within recess 410 and retained by flanges 412 and flanges 408. The clinician may visually inspect retainer 400, IMD 108, and excess portion 306 via opening 414. Proximal end 418 of electrical lead 112 is connected to a distal end of IMD 108 and a distal portion of electrical lead 112 extend through distal opening 420 of expandable member 110 and into the vasculature of patient 102.

The clinician may roll expandable member 110 over IMD 108 and excess portion 306 via retainer 400 by exerting force 416 on retainer 400. In some examples the clinician may hold IMD 108 and excess portion 306 within expandable member 110 while exerting force 416 on retainer 400 to retract retainer 400 relative to IMD 108. Retainer 400 may retain expandable member 100 in recesses 406 as retainer 400 is retracted relative to IMD 108. In some examples, as clinician retracts retainer 400 relative to IMD 108, retainer 400 causes expandable member 100 to roll over and fully envelop IMD 108 and excess portion 306.

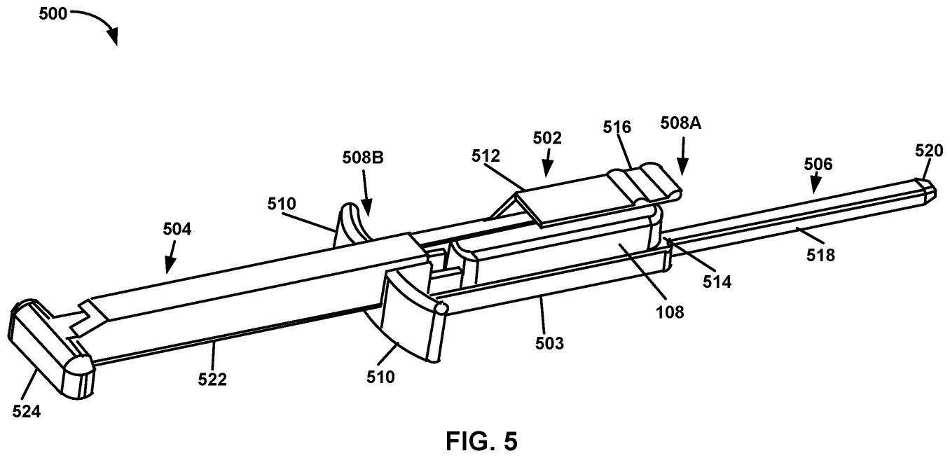

FIG. 5 is a conceptual diagram illustrating another example retainer 500 of lead management system 106. Retainer 500 may include retainer body 502, plunger 504, and tunneling tool 506. Tunneling tool 506 may be configured to create a tunnel from the outer surface of skin of patient 102 into the subcutaneous pocket of patient 102. Tunneling tool 506 may be further configured to guide expandable member 110 into the subcutaneous pocket. Plunger 504 may engage recess 514 (also referred to as “channel 514”) of retainer body 502 and insert IMD 108 and/or excess portion 306 into an inner volume of expandable member 110.

Retainer body 502 may include an elongated body 503 and extension 512 protruding from elongated body and extending towards distal end 508A. Elongated body 503 and extension 512 may define recess 514. Recess 514 may be configured to retain IMD 108 and Excess portion 306 of electrical lead 112 (not pictured). Extension 512 may define recess 516 configured to retain expandable member 110 in a pre-deployed configuration around retainer body 502.

Retainer body 502 may include flanges 510 disposed on proximal end 508B of retainer body 502. Flanges 510 may facilitate movement of plunger 504 along recess 514. Plunger 504 may include shaft 522 and handle 524. Shaft 522 may be configured to engaged with and move into/out of recess 514.

Tunneling tool 506 includes tunneling body 518 with distal end 520. Tunneling body 518 and/or distal end 520 may be tapered to expand and/or dilate a tunnel within tissue of patient 102 to receive IMD 108, expandable member 110, and excess portion 306. In some examples, tunneling tool 506 may form the tunnel from an incision made by the clinician at the insertion site and into the vasculature of patient 102. Tunneling tool 506 may be permanently connected to or removably connected to retainer body 502.

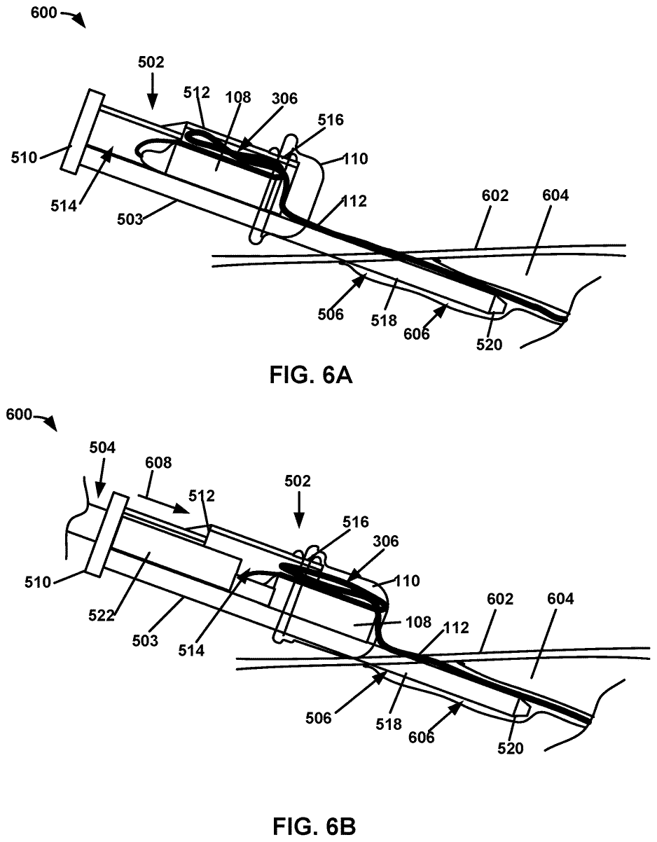

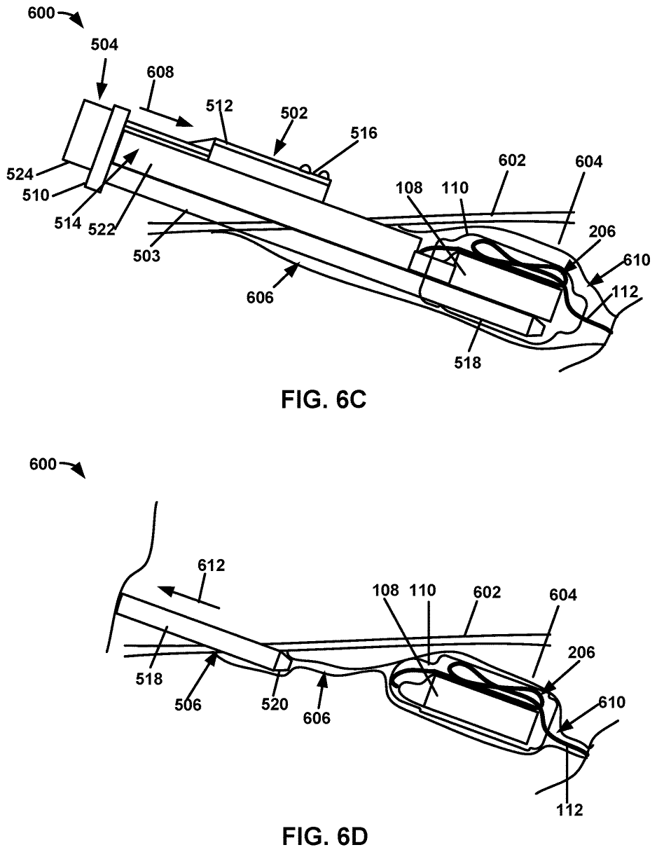

FIG. 6A-D is a conceptual diagram illustrating an example process of implanting expandable member 110 and IMD 108 into tissue of patient 102 via retainer 500 of FIG. 5. FIG. 6A illustrates retainer 500 retaining expandable member 110 in a pre-deployed configuration. FIG. 6B illustrates retainer 500 retaining expandable member 110 in a partially deployed configuration. FIG. 6C illustrates retainer 500 placing deployed expanded member 110 inside tissue of patient 102. FIG. 6D illustrates retraction of retainer 500 after disposal of expanded member 110 within tissue of patient 102.

As illustrated in FIG. 6A, the physician may place expandable member 110 in recess 516 in an pre-deployed configuration. A portion of retainer body 503, IMD 108, and excess portion 306 may be placed within expandable member 110 while expandable member 110 is in the pre-deployer configuration. IMD 108 and excess portion 306 may be disposed within recess 514. An end (e.g., distal end 302A) of IMD 108 may be in contact with a distal end of shaft 522 of plunger 504.

Tunneling body 518 of tunneling tool 506 may be disposed within tunnel 606. Tunnel 606 may be an incision created by the physician in skin 602 and tissue 604 of patient 102 may lead to the vasculature of patient 102.

As illustrated in FIG. 6B, the clinician may exert force 608 on plunger 504 to advance shaft 522 distally within recess 514. Shaft 522 may push IMD 108 and excess portion 306 into the inner volume of expandable member 110. Expandable member 110 may expand due to insertion of IMD 108 and excess portion 306. A proximal end of expandable member 110 may be retained by recess 516 as IMD 108 and excess portion 306 are inserted within expandable member 110.

Once plunger 504 fully inserts IMD 108 and excess portion 306 within expandable member 110, the clinician may continue to exert force 608 onto plunger 504 to insert expandable member 110, IMD 108, and excess portion 306 within subcutaneous pocket 610 via tunnel 606. As expandable member 110 is advanced along tunnel 606, the proximal end of expandable member 110 may detach from recess 516 and contract. As illustrated in FIG. 6C, plunger 504 may advance along tunneling body 518 to dispose expandable member 110 within subcutaneous pocket 610. Once plunger 504 reaches the end of travel within recess 514 (e.g., when handle 524 contacts flanges 510), expandable member 110 may detach from retainer 500 and be disposed within subcutaneous pocket 610. In some examples, the clinician may manually exert a force on expandable member 110 to detach expandable member 100 from retainer 500 (e.g., from tunneling body 518). In other examples, expandable member 110 may detach automatically.

Once the clinician has disposed expandable member 110 within subcutaneous pocket 610, the clinician may exert force 612 on retainer 500 to retract retainer 500 proximally from tunnel 606 while leaving expandable member 110, IMD 108, and electrical lead 112 within subcutaneous pocket 610 of patient 102.

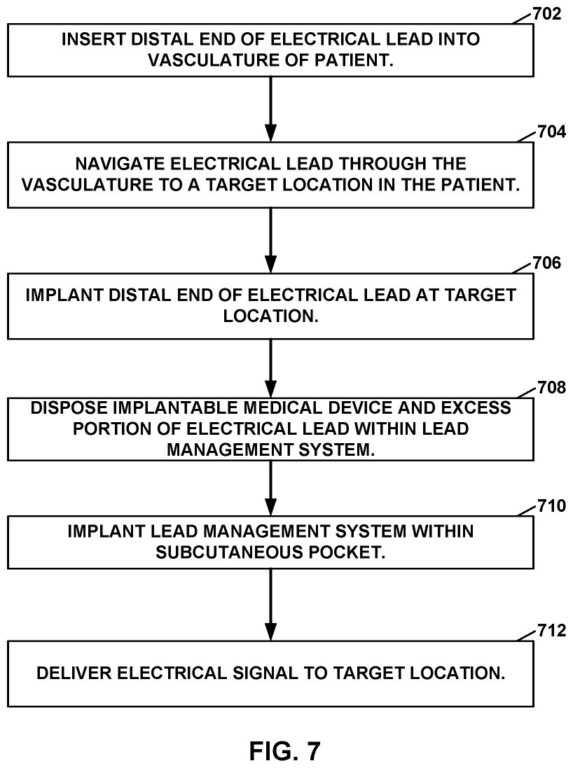

FIG. 7 is a flowchart illustrating an example process of delivering electrical signals to a patient. A clinician may insert distal end 114 of an electrical lead 112 into vasculature of patient 102 (702). The clinician may make an insertion at an insertion site on patient 102. The insertion may extend from the skin of patient 102 into vasculature of patient 102, e.g., via a blood vessel of patient 102. Once the clinician makes the incision, the clinician may insert distal end 114 of electrical lead 112 through the incision and into the vasculature.

The clinician may navigate electrical lead 112 through the vasculature to a target location (also referred to as “target treatment site”) within patient 102 (704). The target location may be any location within vasculature of patient 102 that the clinician wants to deliver electrical energy to. In some examples, the target location may include wall tissue (e.g., at the Triangle of Koch) of one or more chambers of heart 104 of patient 102 (e.g., the right atrium of patient 102, the right ventricle of patient 102). The clinician may navigate electrical lead 112 to the target location via one or more imaging techniques (e.g., X-ray imaging, fluoroscopy, ultrasound or the like). In some examples, the clinician may navigate electrical lead 112 via one or more radiopaque markers disposed on electrical lead 112.

The clinician may implant distal end 114 of electrical lead 114 at the target location (706). The clinician may implant distal end 114 of electrical lead 112 via one or more fixation features disposed on distal end 114. Fixation features may include, but are not limited, penetrator(s) (e.g., a helical penetrator), tines, adhesives, or the like.

The clinician may dispose IMD 108 and excess portion 306 of electrical lead 112 within lead management system 106 (708). Lead management system 106 includes lead management device 110 such as expandable member 110. The clinician may dispose IMD 108 and excess portion 306 within an inner volume 304 of expandable member 110. The clinician may arrange excess portion 306 into a storage configuration before placing excess portion 306 into inner volume 304. In some examples, the clinician may use a retainer (e.g., retainer 400 or retainer 500) to dispose IMD 108 and excess portion 306 within inner volume 304. The clinician may inspect expandable member 110 to determine if excess portion 306 is disposed within inner volume 304 such that a portion of excess portion 306 is twisted, crimped, pinched, or the like. Based on a determination that excess portion 306 is twisted, crimped, pinched, or the like, the clinician may reposition excess portion 306 within inner volume 304 and/or reinsert IMD 108 and/or excess portion 306 into expandable member 110.

The clinician may implant lead management system 106 within subcutaneous pocket 610 in patient 102 (710). In some examples, the clinician may implant expandable member 110 within subcutaneous pocket 610 after disposing IMD 108 and excess portion 306 within inner volume 304 of expandable member 110. In other examples, the clinician may implant expandable member 110 within subcutaneous pocket 610 as a part of disposing IMD 108 and excess portion 306 within subcutaneous pocket 610. In some examples, the clinician may create subcutaneous pocket 610 and then dispose expandable member 110 within subcutaneous pocket 610. In other examples, the clinician may create subcutaneous pocket 610 as a part of disposing expandable member 110 within tissue of patient 102.

The clinician may deliver electrical signals to target location (712). In some examples, the clinician may instruct IMD 108 to deliver electrical signals to target location via electrical lead 112 after implantation of IM 108 and electrical lead 112 within tissue of patient 102. In some examples, after implantation of IMD 108, IMD 108 may automatically transmit electrical signals to the target location.

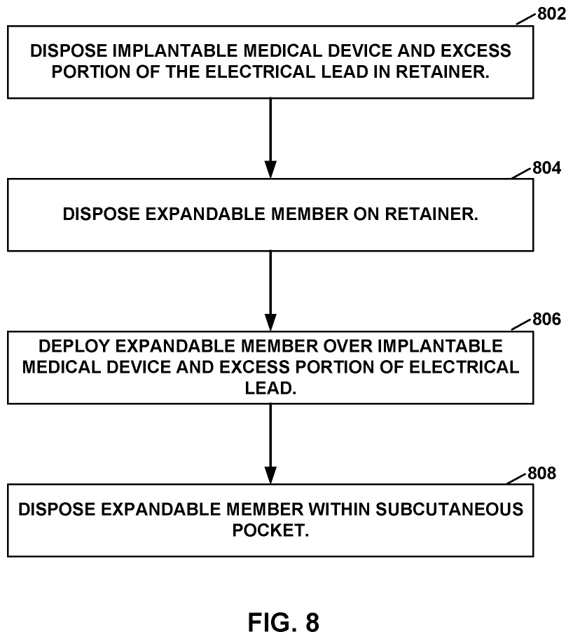

FIG. 8 is a flowchart illustrating an example process of implanting an expandable member and an implantable medical device in a patient. The clinician may dispose IMD 108 and excess portion 306 of electrical lead 112 in a retainer (e.g., retainer 400, retainer 500, or the like) (802). The clinician may dispose IMD 108 and excess portion 306 within a recess (e.g., recess 410, recess 514, or the like) on a body of the retainer (804). The clinician may dispose expandable member 110 on the retainer in a pre-deployed configuration. In some examples, the clinician may dispose expandable member 110 in a second recess (e.g., recess 406, recess 516, or the like) on the body of the retainer.

The clinician may deploy expandable member 110 over IMD 108 and excess portion 306 of electrical lead 112 (806). In some examples, the clinician may hold IMD 108, excess portion 306, and expandable member 110 together as the clinician retracts the retainer (e.g., retainer 400) proximal to IMD 108 to roll expandable member 110 over IMD 108 and excess portion 306. Retainer 400 may retain a proximal end of expandable member 110 as the clinician retracts retainer 400 proximal to IMD 108. Once expandable member 110 is fully deployed over IMD 108 and excess portion 306, retainer 400 may release expandable member 110 and allow expandable member 110 to close. In some examples, the clinician may exert a force on a plunger (e.g., plunger 504) of retainer (e.g., retainer 500) to advance plunger 504 through recess 516 within retainer body 502 to advance IMD 108 and excess portion 306 into inner volume 304 of expandable member 110. Once the clinician fully advances IMD 108 and excess portion 306 into inner volume 304, the clinician may retract plunger 504 to allow expandable member 110 to close and isolate IMD 108 and excess portion 306.

The clinician may dispose expandable member 110 within subcutaneous pocket 610 in patient 102 (808). In some examples, the clinician may dispose a partially deployed expandable member 110, IMD 108, and excess portion 306 within subcutaneous pocket (e.g., via plunger 504). In some examples, the clinician may allow expandable member 110 to fully deploy and close before implanting expandable member 110 within subcutaneous pocket 610.

Electrical lead 112 may be implanted at target location of patient 102 prior to disposal within expandable member 110 and disposal within subcutaneous pocket 610. In other examples, electrical lead 112 may be inserted into vasculature of patient 102 and/or implanted at target location of patient 102 after disposal of IMD 108 and electrical lead 112 within expandable member 110 and prior to disposal of expandable member 110 within subcutaneous pocket 610.

The techniques of this disclosure may be implemented in a wide variety of computing devices, medical devices, or any combination thereof. Any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The disclosure contemplates computer-readable storage media comprising instructions to cause a processor to perform any of the functions and techniques described herein. The computer-readable storage media may take the example form of any volatile, non-volatile, magnetic, optical, or electrical media, such as a RAM, ROM, NVRAM, EEPROM, or flash memory that is tangible. The computer-readable storage media may be referred to as non-transitory. A server, client computing device, or any other computing device may also contain a more portable removable memory type to enable easy data transfer or offline data analysis.

The techniques described in this disclosure, including those attributed to various modules and various constituent components, may be implemented, at least in part, in hardware, software, firmware or any combination thereof. For example, various aspects of the techniques may be implemented within one or more processors, including one or more microprocessors, DSPs, ASICs, FPGAs, or any other equivalent integrated, discrete logic circuitry, or other processing circuitry, as well as any combinations of such components, remote servers, remote client devices, or other devices. The term “processor” or “processing circuitry” may generally refer to any of the foregoing logic circuitry, alone or in combination with other logic circuitry, or any other equivalent circuitry.

Such hardware, software, firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. For example, any module described herein may include electrical circuitry configured to perform the features attributed to that particular module, such as fixed function processing circuitry, programmable processing circuitry, or combinations thereof.

The techniques described in this disclosure may also be embodied or encoded in an article of manufacture including a computer-readable storage medium encoded with instructions. Instructions embedded or encoded in an article of manufacture including a computer-readable storage medium encoded, may cause one or more programmable processors, or other processors, to implement one or more of the techniques described herein, such as when instructions included or encoded in the computer-readable storage medium are executed by the one or more processors. Example computer-readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or any other computer readable storage devices or tangible computer readable media. The computer-readable storage medium may also be referred to as storage devices.

In some examples, a computer-readable storage medium comprises non-transitory medium. The term “non-transitory” may indicate that the storage medium is not embodied in a carrier wave or a propagated signal. In certain examples, a non-transitory storage medium may store data that can, over time, change (e.g., in RAM or cache).

It should be noted that medical device system 100, and the techniques described herein, may not be limited to use in a human patient. In alternative examples, medical device system 100 may be implemented in non-human patients, e.g., primates, canines, equines, pigs, and felines. These other animals may undergo clinical or research therapies that my benefit from the subject matter of this disclosure. Various examples are described herein, such as the following examples.

Example 1: a system comprising: an electrical lead; an implantable medical device, wherein the electrical lead is configured to be electrically connected to the implantable medical device, and the implantable medical device is configured to deliver an electrical therapy to tissue of a patient via the electrical lead; and an expandable member configured to be disposed over the implantable medical device and an excess portion of the electrical lead, wherein the expandable member configured to control movement of the electrical lead within vasculature of the patient.

Example 2: the system of example 1, wherein the expandable member is configured to control the movement of the electrical lead by securing the excess portion of the electrical lead within the expandable member in a storage configuration.

Example 3: the system of any of examples 1 and 2, wherein the expandable member is configured to isolate the implantable medical device and the excess portion of the electrical lead from the tissue of the patient.

Example 4: the system of any of examples 1-3, wherein the expandable member is configured to roll over the implantable medical device from a first end of the implantable medical device to a second end of the implantable medical device.

Example 5: the system of example 4, wherein the first end is a distal end of the implantable medical device, and wherein the second end is a proximal end of the implantable medical device.

Example 6: the system of example 4, wherein the first end is a proximal end of the implantable medical device, and wherein the second end is a distal end of the implantable medical device.

Example 7: the system of any of examples 1-6, wherein the expandable member comprises silicone.

Example 8: the system of any of examples 1-7, wherein the expandable member comprises a biocompatible polymer.

Example 9: the system of any of examples 1-8, wherein the expandable member comprises a coating configured to increase lubricity of the expandable member.

Example 10: the system of any of examples 1-9, wherein the expandable member further comprises an anti-infection material.

Example 11: the system of any of examples 1-10, wherein the expandable member comprises an anti-bleeding and anti-coagulation material.

Example 12: the system of any of examples 1-11, wherein the implantable medical device, the expandable member, and the excess portion of the electrical lead are configured to be implanted into a subcutaneous pocket outside of the vasculature.

Example 13: the system of any of examples 1-12, wherein the expandable member is configured to retain the excess portion of the electrical lead without twisting the electrical lead.

Example 14: the system of any of examples 1-13, further comprising a retainer configured to dispose the expandable member over the implantable medical device and the excess portion of the electrical lead.

Example 15: the system of example 14, wherein the retainer comprises a first recess extending from a proximal end of the retainer to a distal end of the retainer, the first recess configured to retain the implantable medical device and the excess portion of the electrical lead in a storage configuration.

Example 16: the system of any of examples 14 and 15, wherein the retainer comprises a second recess configured to retain the expandable member in a pre-deployed configuration at a distal end of the retainer.

Example 17: the system of any of examples 14-16, wherein a force causing proximal movement of the retainer relative to the implantable medical device causes the expandable member to deploy from a pre-deployed configuration to a deployed configuration, wherein when the expandable member is in the deployed configuration, the expandable member is disposed over the implantable medical device and the excess portion of the electrical lead.

Example 18: the system of any of examples 14-17, wherein the retainer comprises: a device retaining tool comprising: a first recess configured to retain the implantable medical device and the excess portion of the electrical lead in a storage configuration; and a second recess configured to retain the expandable member in a pre-deployed configuration; and a device insertion tool configured to enter the first recess and insert the implantable medical device into the expandable member retained in the second recess.

Example 19: the system of example 18, further comprising a tunneling tool disposed on a distal end of the device retaining tool, the tunneling tool configured to create a subcutaneous pocket within the patient.

Example 20: the system of any of examples 1-19, wherein the expandable member further comprises an anti-rotation feature configured to prevent rotation of the implantable medical device within the expandable member.

Example 21: a lead management device comprising: an expandable member; and an inner volume defined by the expandable member, the inner volume configured to retain an implantable medical device configured to deliver an electrical therapy to tissue of a patient and an excess portion of an electrical lead electrically connected to the implantable medical device, wherein the expandable member is configured to control movement of the electrical lead within vasculature of the patient.

Example 22: the device of example 21, wherein the lead management device is configured to control the movement of the electrical lead by securing the excess portion of the electrical lead within the expandable member in a storage configuration.

Example 23: the device of any of examples 21 and 22, wherein the lead management device is configured to isolate the implantable medical device and the excess portion of the electrical lead from the tissue of the patient.

Example 24: the device of any of examples 21-23, wherein the expandable member is configured to roll over the implantable medical device from a distal end of the implantable medical device to a proximal end of the implantable medical device.

Example 25: the device of example 24, wherein the first end is a distal end of the implantable medical device, and wherein the second is a proximal end of the implantable medical device.

Example 26: the device of example 24, wherein the first end is a proximal end of the implantable medical device, and wherein the second end is a distal end of the implantable medical device.

Example 27: the device of any of examples 21-26, wherein the expandable member comprises silicone.

Example 28: the device of any of examples 21-27, wherein the expandable member comprises a biocompatible polymer.

Example 29: the device of any of examples 21-28, wherein the lead management device further comprises a coating disposed over an outer surface of the expandable member, the coating configured to increase lubricity of the expandable member.

Example 30: the device of any of examples 21-29, wherein the lead management device further comprises an anti-infection material disposed over an inner surface of the expandable member.

Example 31: the device of any of examples 21-30, wherein the lead management device is configured to be implanted within a subcutaneous pocket within the patient.

Example 32: the device of any of examples 21-31, wherein the lead management device is configured to retain the excess portion of the electrical lead without twisting the electrical lead.

Example 33: the device of any of examples 21-32, wherein the lead management device is configured to be disposed over the implantable medical device and the excess portion of the electrical lead via a lead retainer.

Example 34: the device of any of examples 21-33, wherein the expandable member further comprises an anti-rotation feature configured to prevent rotation of the implantable medical device within the expandable member.

Example 35: the device of any of examples 21-34, wherein the expandable member comprises an anti-bleeding and anti-coagulation material disposed over an outer surface of the expandable member.

Example 36: a method comprising: inserting a distal portion of an electrical lead into vasculature of a patient; navigating the distal portion of the electrical lead to a target location within the vasculature of the patient; implanting the distal portion of the electrical lead in tissue of the patient at the target location; disposing an implantable medical device and an excess portion of the electrical lead within an expandable member, wherein the expandable member is configured to control movement of the electrical lead; and delivering electrical therapy to the tissue at the target location through the electrical lead.

Example 37: the method of example 36, wherein disposing the implantable device and the excess portion of the electrical lead within the expandable member comprises: determining the excess portion of the electrical lead; securing the excess portion of the electrical lead into a storage configuration; and disposing the excess portion of the electrical lead in the storage configuration and the implantable medical device within an inner volume of the expandable member.

Example 38: the method of example 37, wherein securing the excess portion of the electrical lead into the storage configuration comprises securing the excess portion of the electrical lead into one or more loops without twisting the electrical lead.

Example 39: the method of any of examples 36-38, wherein the expandable member isolates the implantable medical device and the excess portion of the electrical lead from the tissue of the patient.

Example 40: the method of any of examples 36-39, further comprising implanting the expandable member containing the implantable medical device and the excess portion of the electrical lead within a subcutaneous pocket in the tissue of the patient.

Example 41: the method of any of examples 36-40, wherein disposing the implantable medical device and the excess portion of the electrical lead within the expandable member comprises: placing the excess portion of the electrical lead adjacent to the implantable medical device; and rolling the expandable member over the implantable medical device and the excess portion of the electrical lead from a distal end of the implantable medical device to a proximal end of the implantable medical device.

Example 42: the method of any of examples 36-41, wherein the expandable member comprises silicone.

Example 43: the method of any of examples 36-42, wherein the expandable member comprises a biocompatible polymer.

Example 44: the method of any of examples 36-43, wherein the expandable member comprises a coating disposed over an outer surface of the expandable member, the coating being configured to increase lubricity of the outer surface of the expandable member.

Example 45: the method of any of examples 36-44, wherein the expandable member further comprises an anti-infection material.

Example 46: the method of any of examples 36-45, wherein the expandable member further comprises an anti-bleeding and anti-coagulation material.

Example 47: the method of any of examples 36-46, wherein the medical device system further comprises a retainer, and wherein disposing the implantable medical device and the excess portion of the electrical lead within the expandable member comprises: applying, via the retainer, the expandable member over the implantable medical device and the excess portion of the electrical lead.

Example 48: the method of example 47, wherein the retainer comprises: a first recess extending from a proximal end of the retainer to a distal end of the retainer; and a second recess disposed on one or more extensions at the distal end of the retainer.

Example 49: the method of example 48, wherein applying, via the retainer, the expandable member over the implantable medical device and the excess portion of the electrical lead comprises: disposing the implantable medical device and the excess portion of the electrical lead in the first recess; disposing the expandable member in a pre-deployed configuration in the second recess; and deploying the expandable member over the implantable medical device and the excess portion of the electrical lead via proximal movement of the retainer relative to the implantable medical device.

Example 50: the method of any of examples 47-49, further comprising inserting, via the retainer, the implantable medical device and the excess portion of the electrical lead into a subcutaneous pocket in the tissue of the patient.

Example 51: the method of example 50, wherein the retainer comprises a tunneling tool disposed on a distal end of the retainer, and wherein inserting the implantable medical device and the excess portion of the electrical lead into the subcutaneous pocket comprises: forming, via the tunneling tool, the subcutaneous pocket within the tissue of the patient.

Example 52: the method of any of examples 36-51, wherein the expandable member further comprises an anti-rotation feature configured to prevent rotation of the implantable medical device within the expandable member.

Various examples have been described herein. Any combination of the described operations or functions is contemplated. These and other examples are within the scope of the following claims.

1. A system comprising:
an electrical lead;
an implantable medical device, wherein the electrical lead is electrically connected to the implantable medical device, and wherein the implantable medical device is configured to deliver an electrical therapy to tissue of a patient via the electrical lead; and
an expandable member configured to be disposed over the implantable medical device and an excess portion of the electrical lead, wherein the expandable member is configured to control movement of the electrical lead within vasculature of the patient.

2. The system of claim 1, wherein the expandable member is configured to isolate the implantable medical device and the excess portion of the electrical lead from the tissue of the patient.

3. The system of claim 1, wherein the expandable member is configured to roll over the implantable medical device from a first end of the implantable medical device to a second end of the implantable medical device.

4. (canceled)

5. The system of claim 1, wherein the expandable member comprises a biocompatible polymer.

6-8. (canceled)

9. The system of claim 1, further comprising a retainer configured to dispose the expandable member over the implantable medical device and the excess portion of the electrical lead.

10. The system of claim 9, wherein the retainer comprises a first recess extending from a proximal end of the retainer to a distal end of the retainer, the first recess configured to retain the implantable medical device and the excess portion of the electrical lead in a storage configuration.

11. The system of claim 9, wherein the retainer comprises a second recess configured to retain the expandable member in a pre-deployed configuration at a distal end of the retainer.

12. The system of claim 9, wherein a force causing proximal movement of the retainer relative to the implantable medical device causes the expandable member to deploy from a pre-deployed configuration to a deployed configuration, wherein when the expandable member is in the deployed configuration, the expandable member is disposed over the implantable medical device and the excess portion of the electrical lead.

13. The system of claim 9, wherein the retainer comprises:
a device retaining tool comprising:
a first recess configured to retain the implantable medical device and the excess portion of the electrical lead in a storage configuration; and
a second recess configured to retain the expandable member in a pre-deployed configuration; and
a device insertion tool configured to enter the first recess and insert the implantable medical device into the expandable member retained in the second recess.

14. The system of claim 13, further comprising a tunneling tool disposed on a distal end of the device retaining tool, the tunneling tool configured to create a subcutaneous pocket within the patient.

15. The system of claim 1, wherein the expandable member further comprises an anti-rotation feature configured to prevent rotation of the implantable medical device within the expandable member.

16. A lead management device comprising:
an expandable member; and
an inner volume defined by the expandable member, the inner volume configured to retain an implantable medical device configured to deliver an electrical therapy to tissue of a patient and an excess portion of an electrical lead electrically connected to the implantable medical device,
wherein the expandable member is configured to control movement of the electrical lead within vasculature of the patient.

17. The lead management device of claim 16, wherein the lead management device is configured to isolate the implantable medical device and the excess portion of the electrical lead from the tissue of the patient.

18. The lead management device of claim 16, wherein the expandable member is configured to roll over the implantable medical device from a first end of the implantable medical device to a second end of the implantable medical device.

19. The lead management device of claim 16, wherein the lead management device further comprises a coating disposed over an outer surface of the expandable member, the coating configured to increase lubricity of the expandable member.

20. A method comprising:
inserting a distal portion of an electrical lead into vasculature of a patient;
navigating the distal portion of the electrical lead to a target location within the vasculature of the patient;
implanting the distal portion of the electrical lead in tissue of the patient at the target location;
disposing an implantable medical device and an excess portion of the electrical lead within an expandable member, wherein the expandable member is configured to control movement of the electrical lead; and
delivering electrical therapy to the tissue at the target location through the electrical lead.

21. The method of claim 20, wherein disposing the implantable device and the excess portion of the electrical lead within the expandable member comprises:
determining the excess portion of the electrical lead;
securing the excess portion of the electrical lead into a storage configuration; and
disposing the excess portion of the electrical lead in the storage configuration and the implantable medical device within an inner volume of the expandable member.

22. The method of claim 20, wherein disposing the implantable medical device and the excess portion of the electrical lead within the expandable member comprises:
placing the excess portion of the electrical lead adjacent to the implantable medical device; and
rolling the expandable member over the implantable medical device and the excess portion of the electrical lead from a distal end of the implantable medical device to a proximal end of the implantable medical device.

23. The method of claim 20, wherein the medical device system further comprises a retainer, and wherein disposing the implantable medical device and the excess portion of the electrical lead within the expandable member comprises:
applying, via the retainer, the expandable member over the implantable medical device and the excess portion of the electrical lead.

24. The method of claim 20,
wherein the retainer comprises:
a first recess extending from a proximal end of the retainer to a distal end of the retainer; and
a second recess disposed on one or more extensions at the distal end of the retainer, and
wherein applying, via the retainer, the expandable member over the implantable medical device and the excess portion of the electrical lead comprises:
disposing the implantable medical device and the excess portion of the electrical lead in the first recess;
disposing the expandable member in a pre-deployed configuration in the second recess; and
deploying the expandable member over the implantable medical device and the excess portion of the electrical lead via proximal movement of the retainer relative to the implantable medical device.
